(19) United States
(12) Patent Application Publication
(10) Pub. No.: US 2017/0240543 A1
(43) Pub. Date: Aug. 24, 2017

(54) CRYSTALLINE FORMS OF PALBOCICLIB

(71) Applicant: Sun Pharmaceutical Industries Limited, Mumbai, Maharashtra (IN)

(72) Inventors: Sangeeta SANGWAN, Bhiwani (IN); Anu ARYA, Muzaffarnagar (IN); Kallimulla MOHAMMAD, Krishna (IN); Bishwa Prakash RAI, Azamgarh (IN); Ram THAIMATTAM, Hyderabad (IN); Mohan PRASAD, Gurgaon (IN)

(21) Appl. No.: 15/504,001

(22) PCT Filed: Aug. 13, 2015
(86) PCT No.: PCT/IB2015/056187

§ 371 Date: Feb. 14, 2017

(30) Foreign Application Priority Data
Aug. 14, 2014 (IN) 2325/DEL/2014

(51) Int. Cl. C07D 471/04

(52) U.S. Cl. CPC C07D 471/04; C07B 2200/13

(57) ABSTRACT

The present invention provides crystalline Form I, Form II, Form III, Form IV, Form V, Form V-A, Form VI, Form VII, and Form VIII of palbociclib, processes for their preparation, pharmaceutical compositions comprising these crystalline forms, and their use for the treatment of cyclin-dependent kinase associated diseases.

### FIELD OF THE INVENTION

The present invention provides crystalline Form I, Form II, Form III, Form IV, Form V, Form V-A, Form VI, Form VII, and Form VIII of palbociclib, processes for their preparation, pharmaceutical compositions comprising these crystalline forms, and their use for the treatment of cyclin-dependent kinase (cdk) associated diseases.

### BACKGROUND OF THE INVENTION

Palbociclib of Formula I is chemically described as 6-acetyl-8-cyclopentyl-5-methyl-2-[[5-(1-piperazinyl)-2-pyridinyl]amino]pyrido[2,3-d]pyrimidin-7(8H)-one.

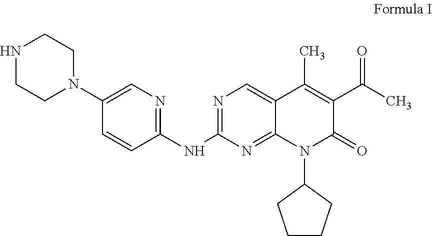 [SMILES: CC(=O)c1c(C)c2cnc(Nc3ccc(N4CCNCC4)cn3)nc2n(C2CCCC2)c1=O]

U.S. Pat. No. 6,936,612 provides a process for the preparation of palbociclib hydrochloride.

U.S. Pat. No. 7,781,583 provides a process for the preparation of palbociclib isoethionate.

U.S. Pat. No. 7,863,278 provides polymorphs of various salts of palbociclib.

PCT Publication No. WO 2014/128588 provides crystalline Forms A and B of palbociclib.

### SUMMARY OF THE INVENTION

The present invention provides crystalline Form I, Form II, Form III, Form IV, Form V, Form V-A, Form VI, Form VII, and Form VIII of palbociclib, processes for their preparation, pharmaceutical compositions comprising these crystalline forms, and their use for the treatment of cyclin-dependent kinase (cdk) associated diseases.

### BRIEF DESCRIPTION OF THE DRAWINGS

FIG. 1 depicts an X-Ray Powder Diffraction (XRPD) pattern of crystalline Form I of palbociclib.

FIG. 2 depicts a Differential Scanning Calorimetry (DSC) thermogram of crystalline Form I of palbociclib.

FIG. 3 depicts an Infra-red (IR) spectrum of crystalline Form I of palbociclib.

FIG. 4 depicts an XRPD pattern of crystalline Form II of palbociclib.

FIG. 5 depicts a DSC thermogram of crystalline Form II of palbociclib.

FIG. 6 depicts an IR spectrum of crystalline Form II of palbociclib.

FIG. 7 depicts an XRPD pattern of crystalline Form III of palbociclib.

FIG. 8 depicts an XRPD pattern of crystalline Form IV of palbociclib.

FIG. 9 depicts an XRPD pattern of crystalline Form V of palbociclib.

FIG. 10 depicts a DSC thermogram of crystalline Form V of palbociclib.

FIG. 11 depicts an IR spectrum of crystalline Form V of palbociclib.

FIG. 12 depicts an XRPD pattern of crystalline Form VI of palbociclib.

FIG. 13 depicts an XRPD pattern of crystalline Form VII of palbociclib.

FIG. 14 depicts a DSC thermogram of crystalline Form VII of palbociclib.

FIG. 15 depicts an XRPD pattern of crystalline Form VIII of palbociclib.

FIG. 16 depicts an XRPD pattern of crystalline Form V-A of palbociclib.

FIG. 17 depicts DSC thermogram of crystalline Form V-A of palbociclib.

FIG. 18 depicts IR spectrum of crystalline Form V-A of palbociclib.

FIG. 19 depicts Thermogravimetric analysis (TGA) thermogram of crystalline Form V-A of palbociclib.

FIG. 20 depicts Scanning electron microscopy (SEM) image of crystalline Form V-A of palbociclib.

### DETAILED DESCRIPTION OF THE INVENTION

The term “about,” as used herein, refers to any value which lies within the range defined by a number up to +10% of the value.

The term “ambient temperature,” as used herein, refers to a temperature in the range of 25° C. to 35° C.

The term “contacting,” as used herein, refers to dissolving, slurrying, stirring, suspending, or combinations thereof.

The term “cyclin-dependent kinase (cdk) associated diseases,” as used herein refers to the diseases mediated by cdk, which include but are not limited to, the cancers of the breast, ovary, cervix, prostate, testis, esophagus, and stomach.

A first aspect of the present invention provides a crystalline form of palbociclib, designated as Form I, characterized by an X-ray powder diffraction (XRPD) pattern having peaks at d-spacings of about 2.8, 3.3, 3.6, 3.9, and 7.2 Å, and additional peaks at d-spacings of about 4.4, 5.3, 11.2, 14.0, and 20.4 Å.

Table 1 provides the d-spacing values (Å), the corresponding 2θ values, and the relative intensity of crystalline Form I of palbociclib.

TABLE 1

| d-spacing | Position | Relative Intensity |
|---|---|---|
| (Å) | (±0.2° 2θ) | (%) |
| 20.4 | 4.3 | 1.0 |
| 14.0 | 6.3 | 1.5 |
| 11.2 | 7.9 | 1.2 |
| 10.4 | 8.5 | 0.9 |
| 7.2 | 12.3 | 2.5 |
| 5.3 | 16.9 | 1.5 |
| 4.4 | 20.3 | 1.8 |
| 3.9 | 22.9 | 1.8 |
| 3.6 | 24.7 | 2.6 |
| 3.3 | 27.4 | 12.8 |
| 2.8 | 31.7 | 100.0 |

Crystalline Form I is characterized by a differential scanning calorimetry (DSC) thermogram having endothermic peaks at about 81.3° C., 155.2° C., 238.0° C., and 265.3° C. and an exothermic peak at about 185.7° C. Crystalline Form I is characterized by an infra-red (IR) absorption spectrum having characteristic peaks expressed in cm⁻¹ at about 3420.2, 3233.7, 2953.9, 2481.6, 1696.9, 1649.5, 1580.9, 1552.2, 1454.5, 1400.0, 1371.7, 1289.4, 1250.4, 1151.1, 1122.0, 1084.4, 1017.6, 923.8, 821.5, 802.0, 745.8, 722.7, 689.0, 631.5, 562.0, and 465.8.

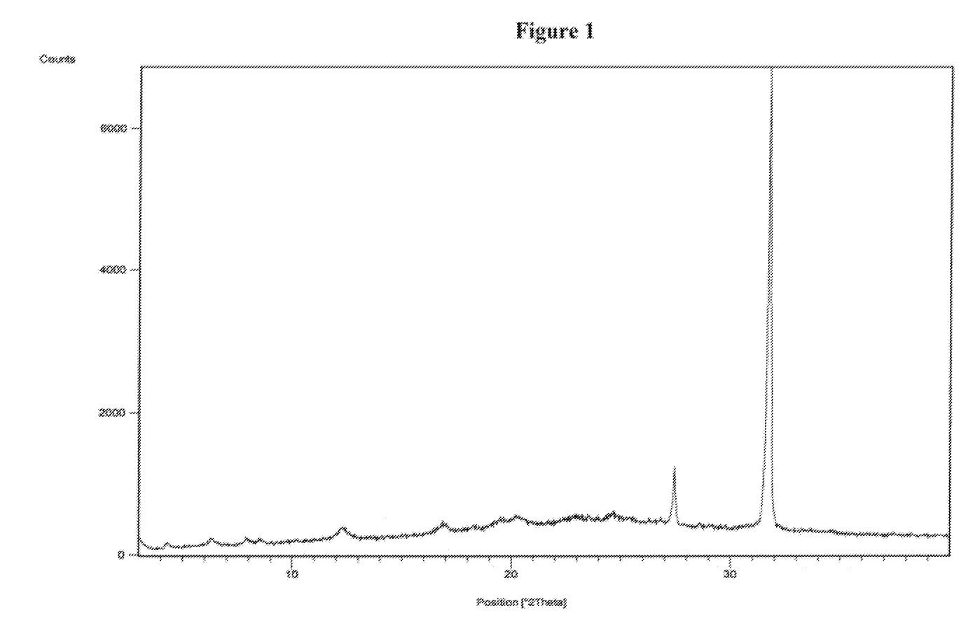

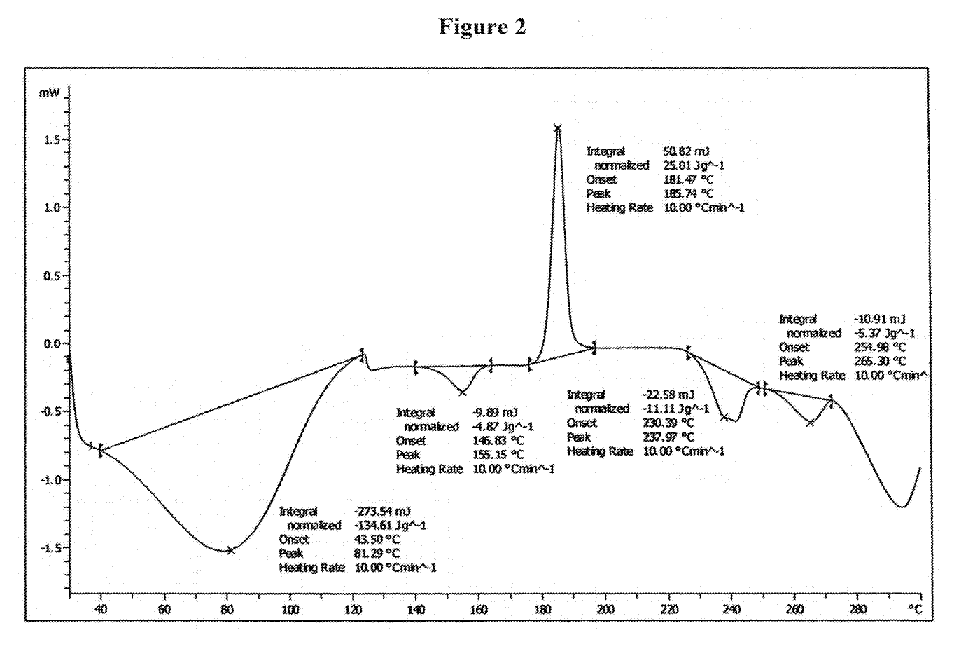

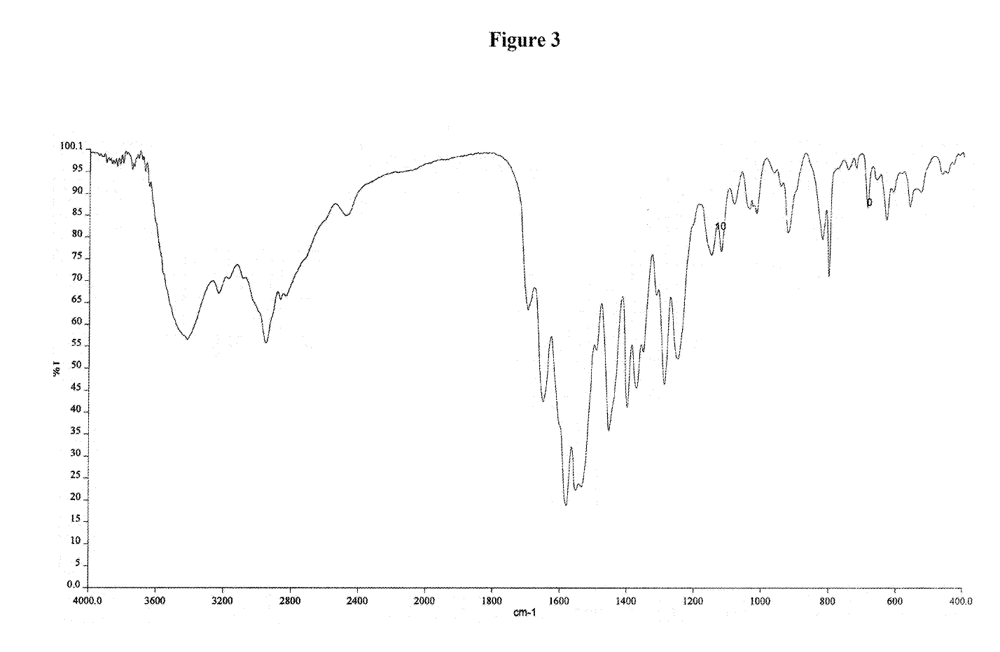

Crystalline Form I of palbociclib is also characterized by an XRPD pattern substantially as depicted in FIG. 1, a DSC thermogram substantially as depicted in FIG. 2, or an IR absorption spectrum substantially as depicted in FIG. 3.

A second aspect of the present invention provides a process for the preparation of crystalline Form I of palbociclib, comprising:
i) contacting palbociclib hydrochloride with a mixture of methanol and water;
ii) adding an acid to the reaction mixture of step i); and
iii) adding a base to the reaction mixture of step ii) to obtain the crystalline Form I of palbociclib.

Palbociclib hydrochloride used for the preparation of crystalline Form I of palbociclib may be prepared by any method provided in the art, for example, the methods as disclosed in U.S. Pat. No. 7,863,278, 6,936,612, or 7,781,583, or by the method as described herein.

Examples of acids include hydrochloric acid, hydrobromic acid, formic acid, propionic acid, methane sulfonic acid, and p-toluene sulfonic acid.

Examples of bases include sodium hydroxide, potassium hydroxide, potassium bicarbonate, sodium carbonate, and triethylamine.

The preparation of crystalline Form I of palbociclib is carried out at ambient temperature for a period of about 30 minutes to about 2 hours, for example, for about 30 minutes to about one hour.

Crystalline Form I of palbociclib may be isolated by filtration, decantation, extraction, distillation, evaporation, chromatography, precipitation, concentration, crystallization, centrifugation, or recrystallization, and dried under reduced pressure, by air drying, or by vacuum tray drying.

A third aspect of the present invention provides a crystalline form of palbociclib, designated as Form II, characterized by an XRPD pattern having peaks at d-spacings of about 2.8, 4.0, 4.5, 5.2, and 8.6 Å. Crystalline Form II of palbociclib is further characterized by an XRPD pattern having additional peaks at d-spacings of about 3.3, 3.9, 4.8, 7.7, and 8.8 Å.

Table 2 provides the d-spacing values (Å), the corresponding 2θ values, and the relative intensity of the crystalline Form II of palbociclib.

TABLE 2

| d-spacing | Position | Relative Intensity |
|---|---|---|
| (Å) | (±0.2° 2θ) | (%) |
| 17.5 | 5.1 | 9.7 |
| 11.1 | 8.0 | 5.8 |
| 8.8 | 10.1 | 15.8 |
| 8.6 | 10.3 | 20.6 |
| 7.7 | 11.5 | 15.3 |
| 6.3 | 14.0 | 3.1 |
| 5.9 | 15.1 | 2.3 |
| 5.6 | 15.9 | 6.6 |
| 5.4 | 16.5 | 1.8 |
| 5.2 | 17.0 | 19.2 |
| 5.1 | 17.4 | 4.6 |
| 5.0 | 17.9 | 3.2 |
| 4.8 | 18.7 | 17.7 |
| 4.5 | 19.6 | 17.9 |
| 4.4 | 20.2 | 5.9 |
| 4.3 | 20.5 | 2.9 |
| 4.2 | 21.1 | 8.4 |
| 4.1 | 21.9 | 7.1 |
| 4.0 | 22.4 | 44.9 |
| 3.9 | 22.9 | 15.5 |
| 3.8 | 23.7 | 5.7 |
| 3.4 | 26.2 | 2.0 |
| 3.3 | 27.4 | 12.8 |
| 3.1 | 28.4 | 7.3 |
| 3.1 | 28.8 | 5.2 |
| 3.0 | 29.4 | 2.3 |
| 3.0 | 30.0 | 3.4 |
| 2.9 | 30.7 | 3.3 |
| 2.8 | 31.7 | 100.0 |
| 2.7 | 32.9 | 1.5 |
| 2.6 | 34.6 | 1.4 |
| 2.5 | 35.8 | 1.3 |
| 2.3 | 39.2 | 1.7 |

Crystalline Form II is characterized by a DSC thermogram having an endothermic peak at about 78.0° C. and an exothermic peak at about 275.1° C. Crystalline Form II is further characterized by an IR absorption spectrum having characteristic peaks expressed in cm⁻¹ at about 3417, 3298, 3232, 3173, 3084, 2947, 2869, 2843, 2469, 1770, 1664, 1602, 1581, 1548, 1528, 1488, 1449, 1396, 1366, 1332, 1310, 1284, 1248, 1235, 1188, 1148, 1122, 1078, 1039, 1022, 978, 937, 924, 899, 873, 860, 827, 805, 795, 776, 758, 747, 737, 720, 689, 646, 627, 617, 572, 533, 457, 431, and 407.

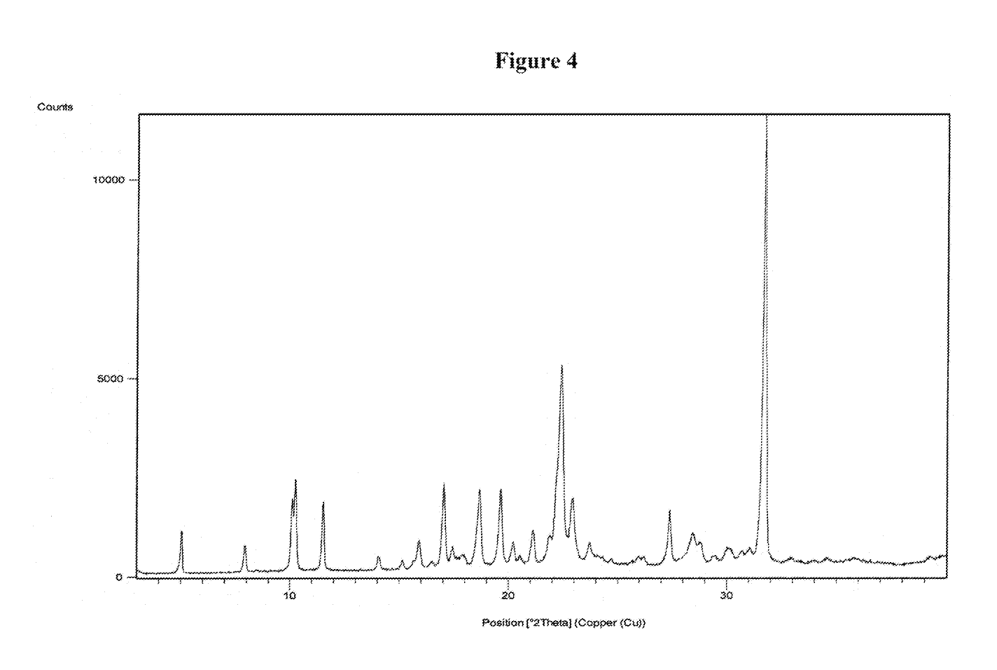

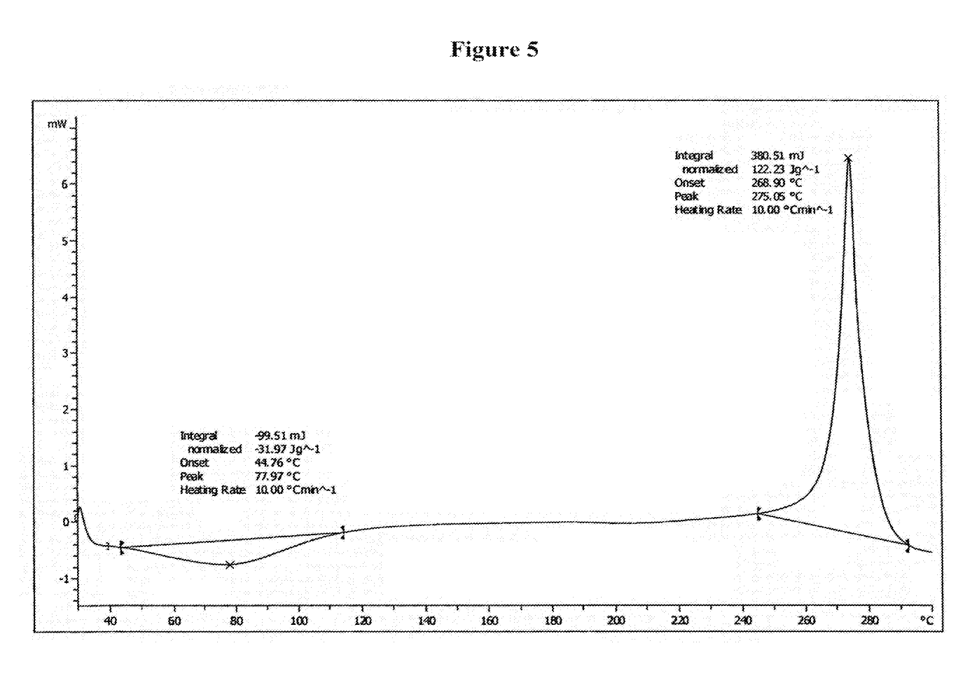

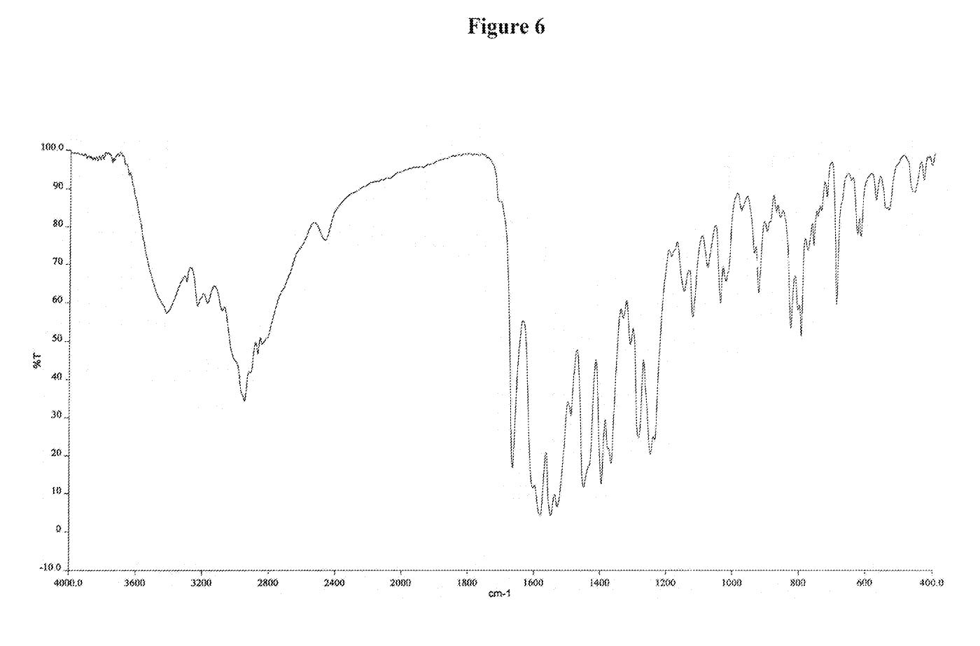

Crystalline Form II of palbociclib is characterized by an XRPD pattern substantially as depicted in FIG. 4, a DSC thermogram substantially as depicted in FIG. 5, or an IR absorption spectrum substantially as depicted in FIG. 6.

A fourth aspect of the present invention provides a process for the preparation of crystalline Form II of palbociclib, comprising:
i) contacting crystalline Form I of palbociclib with a mixture of solvents selected from the group consisting of water and acetone, and water and 2-propanol;
ii) adding hydrochloric acid to the reaction mixture of step i); and
iii) adding sodium hydroxide to the reaction mixture of step ii) to obtain the crystalline Form II of palbociclib.

The preparation of the crystalline Form II of palbociclib is carried out at ambient temperature for a period of about 15 minutes to about 2 hours, for example, for about 20 minutes to about one hour.

Crystalline Form II of palbociclib may be isolated by filtration, decantation, extraction, distillation, evaporation, chromatography, precipitation, concentration, crystallization, centrifugation, or recrystallization. Crystalline Form II of palbociclib may be dried by drying under reduced pressure, by air drying, or by vacuum tray drying.

A fifth aspect of the present invention provides a crystalline form of palbociclib, designated as Form III, characterized by an XRPD pattern having peaks at d-spacings of about 4.0, 5.2, 8.6, and 8.8 Å, and additional peaks at d-spacings of about 3.2, 3.9, 4.5, 4.8, and 7.7 Å.

Table 3 provides the d-spacing values (Å), the corresponding 2θ values, and the relative intensity of the crystalline Form III of palbociclib.

TABLE 3

| d-spacing | Position | Relative Intensity |
|---|---|---|
| (Å) | (±0.2° 2θ) | (%) |
| 17.5 | 5.1 | 24.4 |
| 11.2 | 7.9 | 18.1 |
| 10.5 | 8.5 | 1.1 |
| 8.8 | 10.1 | 54.3 |
| 8.6 | 10.2 | 46.9 |
| 7.7 | 11.5 | 33.1 |
| 6.3 | 14.1 | 7.1 |
| 5.9 | 15.2 | 5.2 |
| 5.6 | 15.8 | 17.9 |
| 5.6 | 15.9 | 12.5 |
| 5.4 | 16.4 | 6.7 |
| 5.2 | 17.0 | 42.3 |
| 5.1 | 17.4 | 14.1 |
| 5.0 | 17.8 | 11.0 |
| 4.8 | 18.7 | 37.4 |
| 4.5 | 19.6 | 38.0 |
| 4.4 | 20.2 | 15.4 |
| 4.2 | 21.0 | 21.6 |
| 4.0 | 22.2 | 97.4 |
| 4.0 | 22.4 | 100.0 |
| 3.9 | 22.9 | 40.9 |
| 3.8 | 23.7 | 16.3 |
| 3.4 | 25.9 | 8.2 |
| 3.3 | 27.2 | 8.2 |
| 3.2 | 28.4 | 24.7 |
| 3.1 | 28.7 | 15.3 |
| 3.0 | 30.0 | 12.1 |
| 2.9 | 30.7 | 9.7 |
| 2.9 | 31.0 | 8.9 |
| 2.7 | 32.8 | 3.5 |
| 2.6 | 34.6 | 3.1 |
| 2.5 | 35.8 | 4.5 |
| 2.3 | 39.2 | 5.2 |

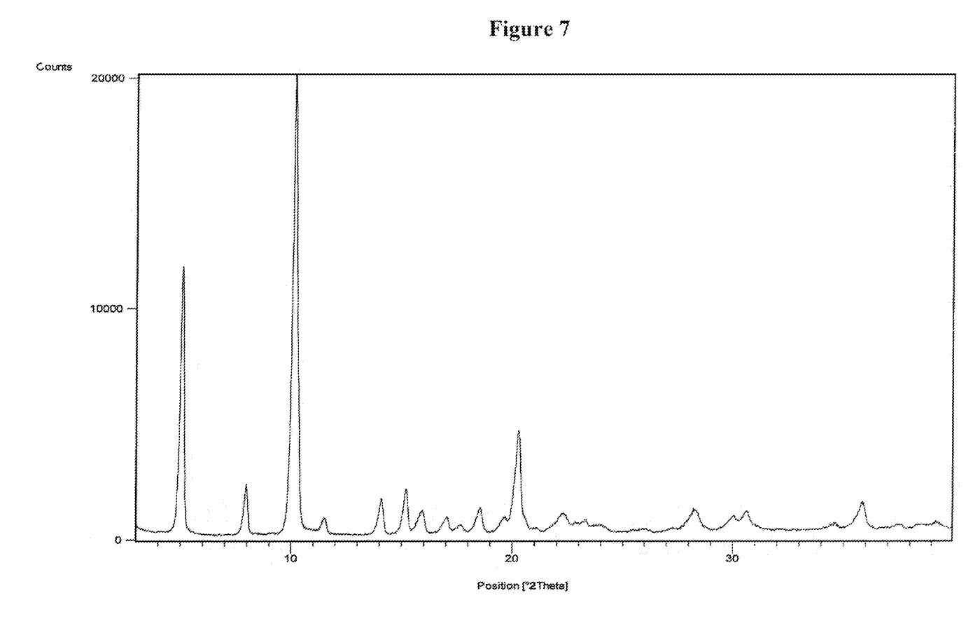

Crystalline Form III of palbociclib is characterized by an XRPD pattern substantially as depicted in FIG. 7.

A sixth aspect of the present invention provides a process for the preparation of crystalline Form III of palbociclib, comprising:
i) contacting crystalline Form I of palbociclib with a mixture of solvents selected from the group consisting of water and 2-propanol, water and acetonitrile, and water and acetone;
ii) adding hydrochloric acid to the reaction mixture of step i); and
iii) adding ammonia to the reaction mixture of step ii) to obtain the crystalline Form III of palbociclib.

The preparation of crystalline Form III of palbociclib is carried out at ambient temperature for a period of about 15 minutes to about 2 hours, for example, for about 25 minutes to about one hour.

Crystalline Form III of palbociclib may be isolated by filtration, decantation, extraction, distillation, evaporation, chromatography, precipitation, concentration, crystallization, centrifugation, or recrystallization. Crystalline Form III of palbociclib may be dried by drying under reduced pressure, by air drying, or by vacuum tray drying.

A seventh aspect of the present invention provides a crystalline form of palbociclib, designated as Form IV, characterized by an XRPD pattern having peaks at d-spacings of about 4.4, 5.8, 8.7, 11.1, and 17.3 Å, and further characterized by an XRPD pattern having additional peaks at d-spacings of about 2.5, 3.2, 4.8, 5.6, and 6.3 Å.

Table 4 provides the d-spacing values (Å), the corresponding 2θ values, and the relative intensity of crystalline Form IV of palbociclib.

TABLE 4

| d-spacing | Position | Relative Intensity |
|---|---|---|
| (Å) | (±0.2° 2θ) | (%) |
| 17.3 | 5.1 | 55.6 |
| 11.1 | 8.0 | 9.7 |
| 8.7 | 10.2 | 100.0 |
| 7.7 | 11.5 | 3.5 |
| 6.3 | 14.1 | 7.7 |
| 5.8 | 15.2 | 10.1 |
| 5.6 | 16.0 | 5.0 |
| 5.2 | 17.1 | 3.7 |
| 5.0 | 17.7 | 1.8 |
| 4.8 | 18.6 | 5.7 |
| 4.5 | 19.7 | 3.5 |
| 4.4 | 20.4 | 19.5 |
| 4.0 | 22.3 | 4.2 |
| 3.8 | 23.3 | 2.7 |
| 3.7 | 24.1 | 1.3 |
| 3.4 | 26.0 | 0.6 |
| 3.2 | 28.2 | 4.7 |
| 3.0 | 30.1 | 3.2 |
| 2.9 | 30.6 | 4.2 |
| 2.6 | 34.6 | 1.6 |
| 2.5 | 35.9 | 6.1 |
| 2.4 | 37.5 | 1.2 |
| 2.4 | 38.3 | 1.2 |
| 2.3 | 39.3 | 1.6 |

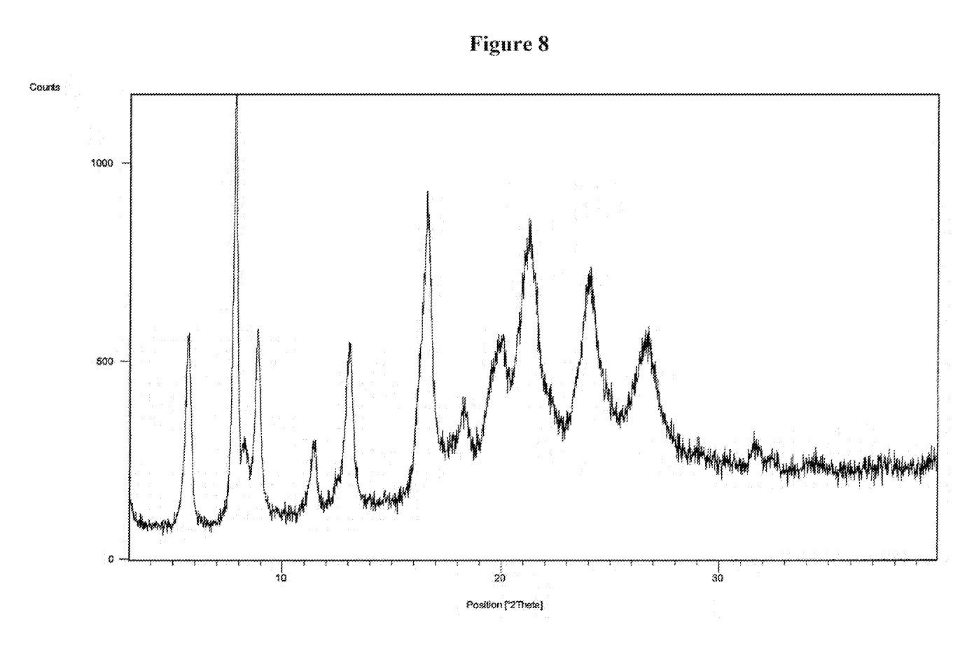

Crystalline Form IV of palbociclib is characterized by an XRPD pattern substantially as depicted in FIG. 8.

An eighth aspect of the present invention provides a process for the preparation of crystalline Form IV of palbociclib, comprising:
i) contacting crystalline Form I of palbociclib with a mixture of water and 1-propanol;
ii) adding hydrochloric acid to the reaction mixture of step i); and
iii) adding ammonia to the reaction mixture of step ii) to obtain the crystalline Form IV of palbociclib.

The preparation of crystalline Form IV of palbociclib is carried out at ambient temperature for a period of about 5 minutes to about one hour, for example, for about 5 minutes to about 30 minutes.

Crystalline Form IV of palbociclib may be isolated by filtration, decantation, extraction, distillation, evaporation, chromatography, precipitation, concentration, crystallization, centrifugation, or recrystallization. Crystalline Form IV of palbociclib may be dried by drying under reduced pressure, by air drying, or by vacuum tray drying.

A ninth aspect of the present invention provides a crystalline form of palbociclib, designated as Form V, characterized by an XRPD pattern having peaks at d-spacings of about 4.2, 5.4, 10.0, 11.3, and 15.6 Å, and further characterized by additional peaks at d-spacings of about 3.3, 3.7, 4.4, 4.9, and 6.8 Å.

Table 5 provides the d-spacing values (Å), the corresponding 2θ values, and the relative intensity of crystalline Form V of palbociclib.

TABLE 5

| d-spacing | Position | Relative Intensity |
|---|---|---|
| (Å) | (±0.2° 2θ) | (%) |
| 15.6 | 5.7 | 44.0 |
| 11.3 | 7.8 | 100.0 |
| 10.0 | 8.9 | 44.2 |
| 7.7 | 11.5 | 14.2 |
| 6.8 | 13.1 | 37.5 |
| 5.4 | 16.6 | 67.9 |
| 4.9 | 18.3 | 18.4 |
| 4.4 | 20.1 | 34.0 |
| 4.2 | 21.2 | 56.3 |
| 3.7 | 24.0 | 42.3 |
| 3.3 | 26.7 | 27.1 |
| 2.8 | 31.6 | 4.5 |

Crystalline Form V of palbociclib is characterized by a DSC thermogram having endothermic peaks at about 62.6° C., 126.0° C., and 262.8° C. Crystalline Form V is further characterized by an IR absorption spectrum having characteristic peaks expressed in cm⁻¹ at about 3418.3, 3231.6, 3166.9, 2947.6, 2865.3, 1691.1, 1655.1, 1585.3, 1552.9, 1489.6, 1453.6, 1398.7, 1376.3, 1349.8, 1315.8, 1283.3, 1233.7, 1159.8, 1122.3, 1082.4, 1039.7, 1015.6, 990.3, 929.9, 906.5, 825.9, 800.9, 748.4, 723.8, 691.0, 623.7, 564.9, 542.6, 447.8, and 430.2.

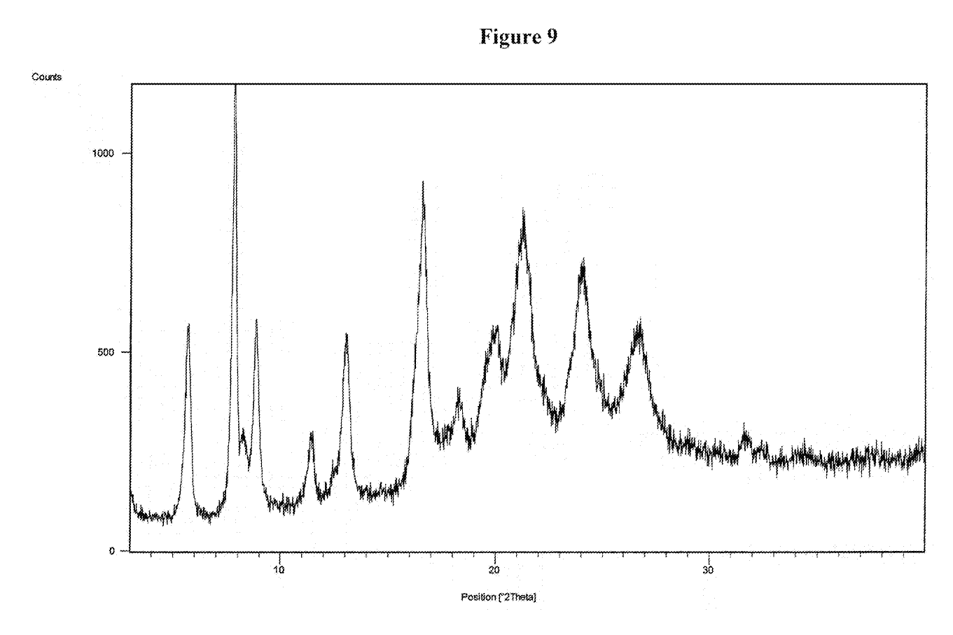

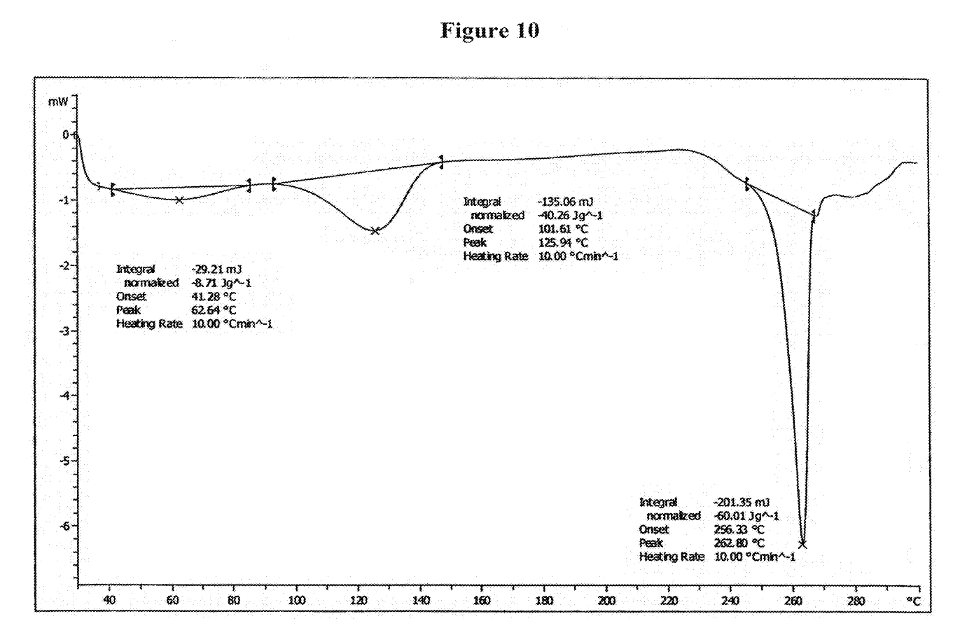

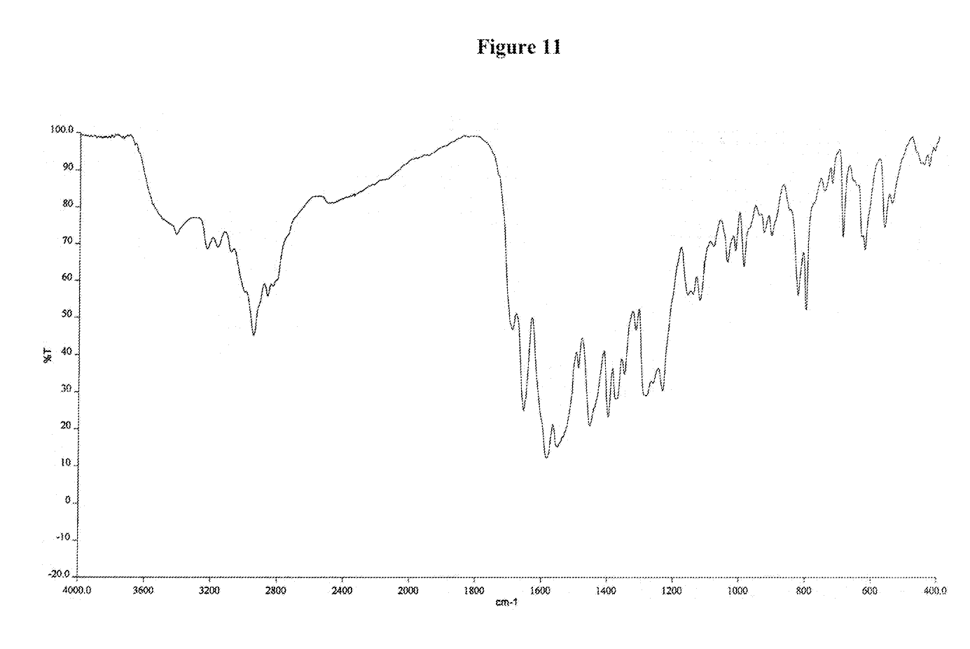

Crystalline Form V of palbociclib is characterized by an XRPD pattern substantially as depicted in FIG. 9, a DSC thermogram substantially as depicted in FIG. 10, or an IR absorption spectrum substantially as depicted in FIG. 11.

A tenth aspect of the present invention provides a process for the preparation of crystalline Form V of palbociclib, comprising:
i) contacting crystalline Form III of palbociclib with a mixture of water and methanol;
ii) adding an inorganic acid selected from the group consisting of sulphuric acid and phosphoric acid to the reaction mixture of step i); and
iii) adding an inorganic base selected from the group consisting of sodium bicarbonate and potassium carbonate to the reaction mixture of step ii) to obtain the crystalline Form V of palbociclib.

The preparation of crystalline Form V of palbociclib is carried out at ambient temperature for a period of about 30 minutes to about 10 hours, for example, for about 30 minutes to about 5 hours.

Crystalline Form V of palbociclib may be isolated by filtration, decantation, extraction, distillation, evaporation, chromatography, precipitation, concentration, crystallization, centrifugation, or recrystallization. Crystalline Form V of palbociclib may be dried by drying under reduced pressure, by air drying, or by vacuum tray drying.

An eleventh aspect of the present invention provides a crystalline form of palbiociclib designated as Form V-A, characterized by an XRPD pattern having peaks at d-spacing of 11.3, 6.8, 5.3, 4.2, and 3.7 Å, and further characterized by additional peaks at d-spacings of about 15.5, 10.0, 4.4, 4.1, and 3.3 Å.

Table 5 Å provides the d-spacing values (Å), the corresponding 2θ values, and the relative intensity of crystalline Form V-A of palbociclib.

TABLE 5A

| d-spacing | Position | Relative Intensity |
|---|---|---|
| (Å) | (±0.2° 2θ) | (%) |
| 15.5 | 5.7 | 40.1 |
| 11.3 | 7.8 | 82.2 |
| 10.7 | 8.3 | 28.3 |
| 10.0 | 8.8 | 44.4 |
| 7.8 | 11.4 | 19.4 |
| 7.1 | 12.4 | 3.8 |
| 6.8 | 13.1 | 66.5 |
| 6.0 | 14.7 | 3.9 |
| 5.5 | 16.1 | 20.9 |
| 5.3 | 16.6 | 100.0 |
| 5.2 | 17.1 | 9.3 |
| 5.0 | 17.6 | 8.5 |
| 4.8 | 18.4 | 23.2 |
| 4.6 | 19.5 | 34.7 |
| 4.4 | 20.0 | 40.3 |
| 4.3 | 20.6 | 31.6 |
| 4.2 | 21.1 | 57.9 |
| 4.1 | 21.4 | 42.7 |
| 4.0 | 22.2 | 17.6 |
| 3.7 | 24.0 | 65.9 |
| 3.6 | 24.9 | 12.1 |
| 3.3 | 26.7 | 54.6 |
| 3.3 | 27.3 | 13.7 |
| 3.2 | 27.8 | 11.3 |
| 3.1 | 29.2 | 10.2 |
| 3.0 | 30.3 | 8.6 |
| 2.8 | 31.5 | 11.7 |
| 2.8 | 32.3 | 9.7 |
| 2.6 | 34.5 | 9.9 |
| 2.5 | 35.4 | 7.2 |
| 2.4 | 37.3 | 9.7 |
| 2.3 | 38.8 | 9.2 |
| 2.3 | 39.6 | 9.3 |

Crystalline Form V-A of palbociclib is characterized by a DSC thermogram having endothermic peaks at about 148.2° C., 269.2° C., 272.1° C., and 284.2° C. and an exothermic peak at about 222.9° C. Crystalline Form V-A of palbociclib is further characterized by an IR absorption spectrum having characteristic peaks expressed in cm⁻¹ at about 3422, 3235, 3168, 2948, 2865, 2804, 2468, 1692, 1654, 1585, 1555, 1488, 1454, 1399, 1377, 1349, 1315, 1293, 1279, 1263, 1233, 1160, 1143, 1125, 1083, 1040, 1016, 990, 931, 906, 826, 801, 749, 724, 691, 635, 624, 567, 543, 445, and 431.

Crystalline Form V-A of palbociclib has a specific surface area (SSA) of greater than 2 m²/g.

In an embodiment, the crystalline Form V-A of palbociclib has a specific surface area (SSA) for example, of about 3 m²/g to about 10 m²/g.

Crystalline Form V-A of palbociclib has a particle size distribution (PSD) having at least one of:
a) a D₁₀ value of about 1 μm to about 5 μm;
b) a D₅₀ value of about 5 μm to about 20 μm;
c) a D₉₀ value of about 15 μm to about 50 μm.

Crystalline Form V-A of palbociclib has a volume mean diameter (D[4,3]) of about 5 μm to about 50 μm.

Crystalline Form V-A of palbociclib has improved filterability, electrostatic nature, and flowability.

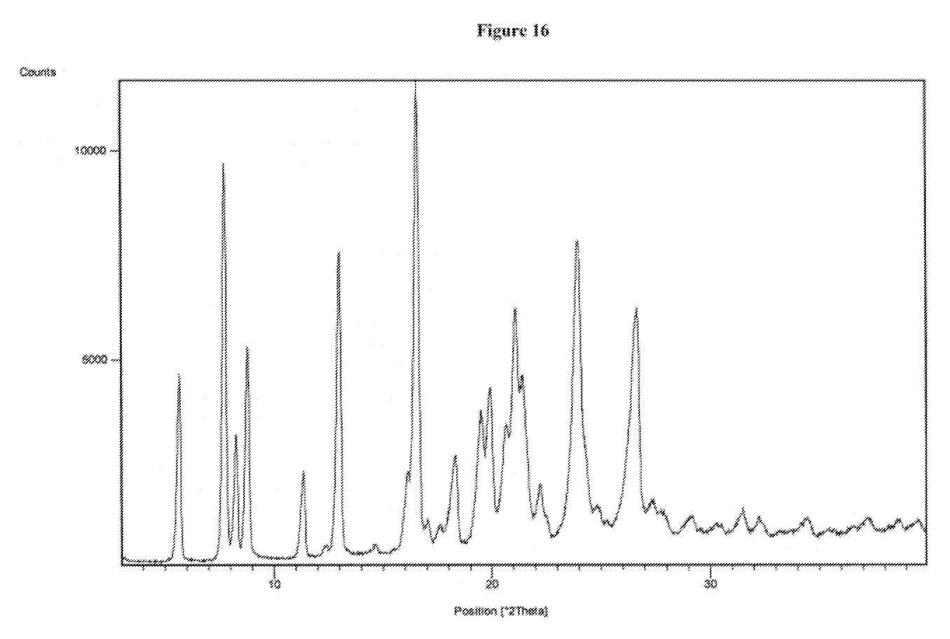

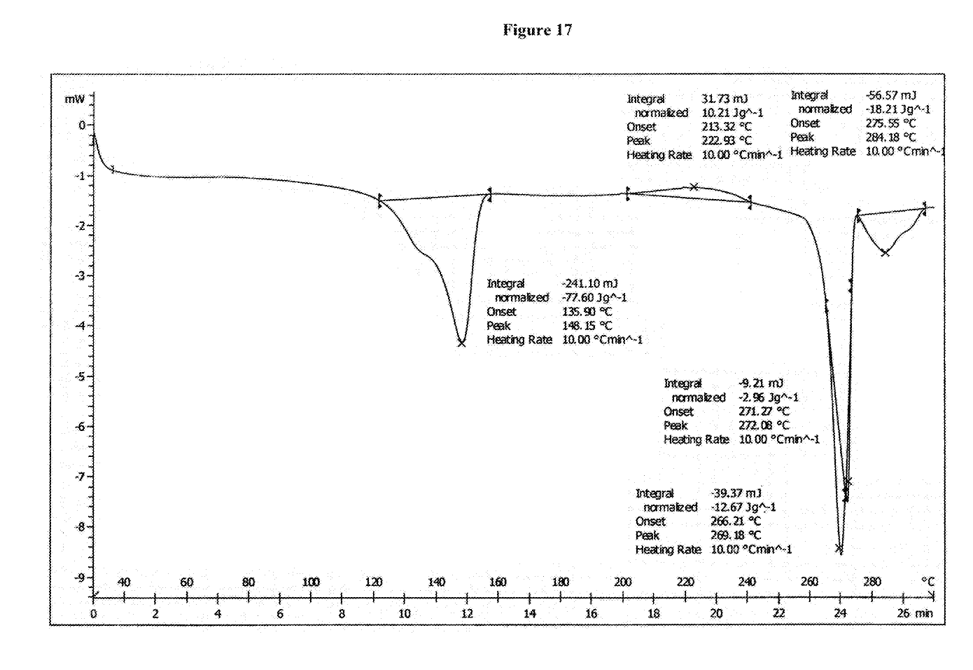

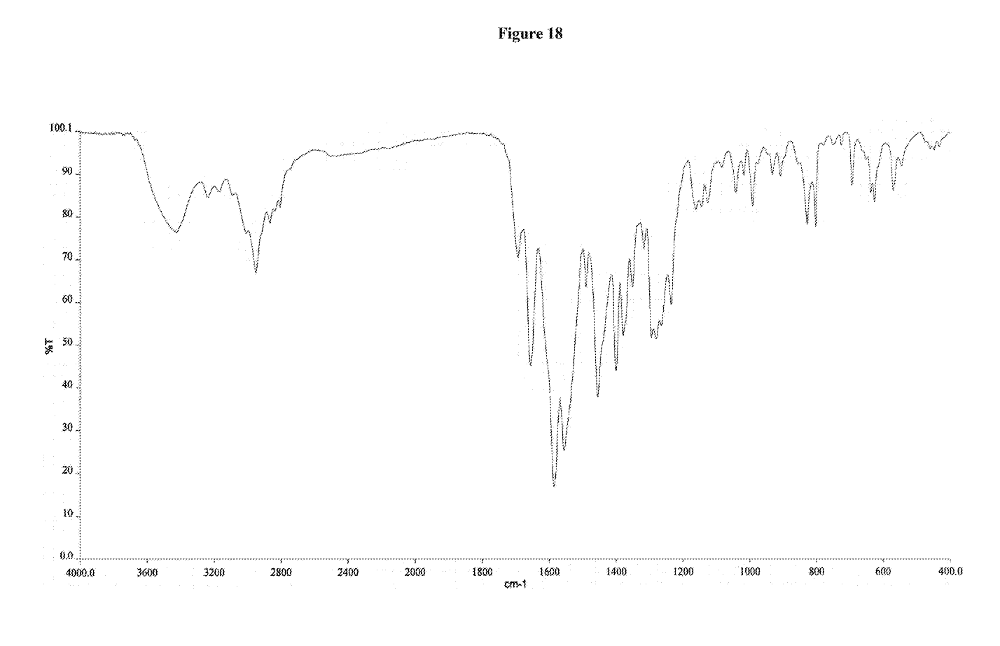

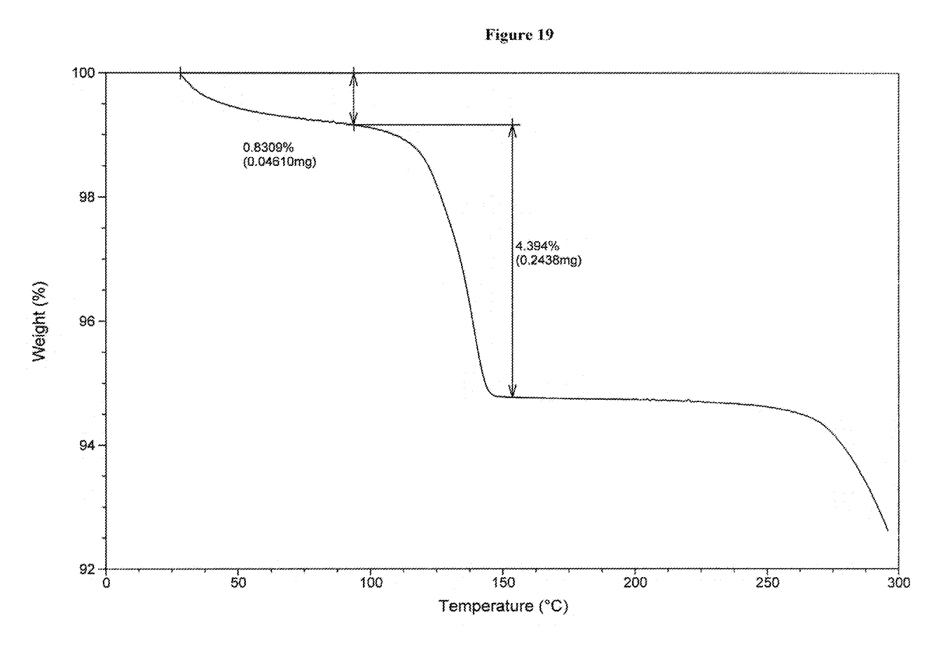

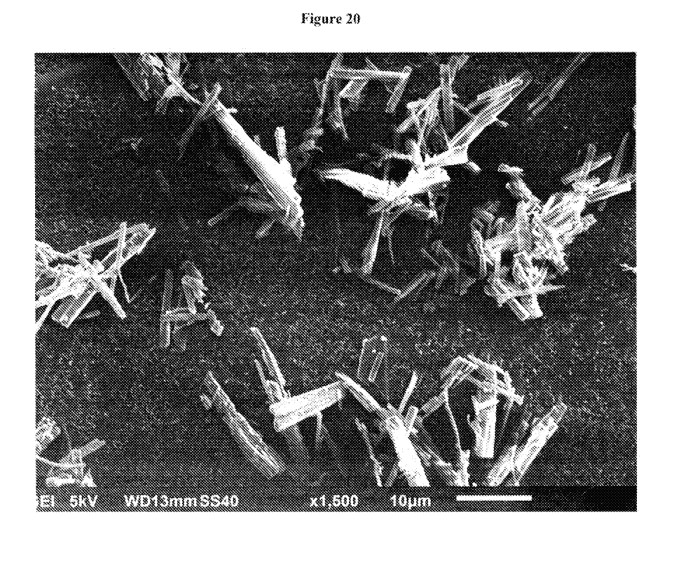

Crystalline Form V-A of palbociclib is characterized by an XRPD pattern substantially as depicted in FIG. 16, a DSC thermogram substantially as depicted in FIG. 17, an IR absorption spectrum substantially as depicted in FIG. 18, a thermogravimetric analysis (TGA) thermogram substantially as depicted in FIG. 19, or a scanning electron microscopy (SEM) image substantially as depicted in FIG. 20.

A twelfth aspect of the present invention provides a process for the preparation of crystalline Form V-A of palbociclib, comprising:
i) contacting palbociclib hydrochloride with a mixture of water and methanol or a mixture of water or acetone; and
ii) adding an inorganic base to the reaction mixture of step (i) to obtain the crystalline Form V of palbociclib.

Palbociclib hydrochloride used for the preparation of crystalline Form V-A of palbociclib may be prepared by any method provided in the art, for example, the method as disclosed in U.S. Pat. No. 7,863,278, 6,936,612, or 7,781,583 or by the method as described herein.

Examples of inorganic bases include sodium bicarbonate, potassium bicarbonate, and calcium bicarbonate.

The preparation of crystalline Form V-A of palbociclib is carried out at about 20° C. to about 55° C. for a period of about 30 minutes to about 5 hours, for example, for about one hour to about 4 hours.

Crystalline Form V-A of palbociclib may be isolated by filtration, decantation, extraction, distillation, evaporation, chromatography, precipitation, concentration, crystallization, centrifugation, or recrystallization. Crystalline Form V-A of palbociclib may be dried by drying under reduced pressure, by air drying, or by vacuum tray drying.

An thirteenth aspect of the present invention provides a crystalline form of palbociclib, designated as Form VI, characterized by an XRPD pattern having peaks at d-spacings of about 3.5, 3.6, 3.7, 4.0, and 4.3 Å and further characterized by additional peaks at d-spacings of about 3.2, 3.4, 4.9, 5.2, and 5.4 Å.

Table 6 provides the d-spacing values (Å), the corresponding 2θ values, and the relative intensity of the crystalline Form VI of palbociclib.

TABLE 6

| d-spacing | Position | Relative Intensity |
|---|---|---|
| (Å) | (±0.2° 2θ) | (%) |
| 15.5 | 5.7 | 1.9 |
| 11.2 | 7.9 | 4.3 |
| 9.8 | 9.0 | 3.0 |
| 8.7 | 10.1 | 9.1 |
| 8.4 | 10.5 | 7.2 |
| 7.6 | 11.6 | 2.2 |
| 6.6 | 13.4 | 19.6 |
| 5.4 | 16.3 | 38.7 |
| 5.2 | 17.0 | 32.1 |
| 4.9 | 18.2 | 31.8 |
| 4.3 | 20.6 | 41.6 |
| 4.0 | 22.1 | 100.0 |
| 3.7 | 23.9 | 53.2 |
| 3.6 | 24.7 | 50.1 |
| 3.5 | 25.3 | 39.8 |
| 3.4 | 25.9 | 37.6 |
| 3.2 | 27.7 | 37.0 |
| 3.0 | 29.9 | 23.0 |
| 2.8 | 32.2 | 14.9 |
| 2.6 | 34.3 | 8.6 |
| 2.5 | 35.5 | 9.1 |
| 2.3 | 39.0 | 5.5 |

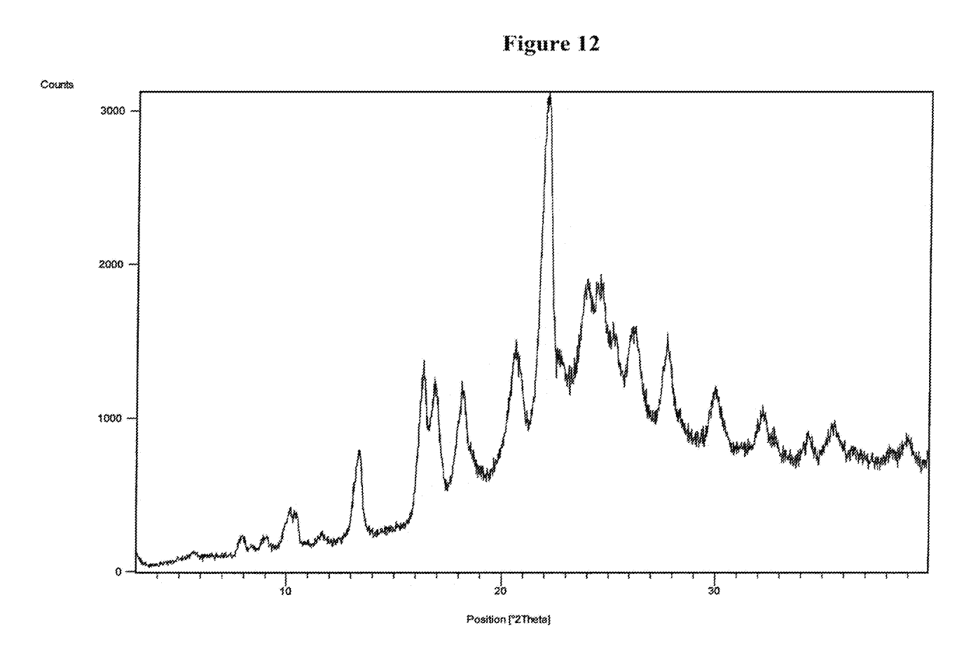

Crystalline Form VI of palbociclib is characterized by an XRPD pattern substantially as depicted in FIG. 12.

A fourteenth aspect of the present invention provides a process for the preparation of crystalline Form VI of palbociclib, comprising contacting crystalline Form III of palbociclib with a solvent selected from the group consisting of 2-propanol, acetone, tetrahydrofuran, and 2-propyl acetate to obtain the crystalline Form VI of palbociclib.

The preparation of the crystalline Form VI of palbociclib is carried out at about 40° C. to about 60° C., for example, at about 40° C. to about 45° C., for about 2 hours to about 6 hours, for example, for about 2 hours to about 4 hours.

The crystalline Form VI of palbociclib may be isolated by filtration, decantation, extraction, distillation, evaporation, chromatography, precipitation, concentration, crystallization, centrifugation, or recrystallization. The crystalline Form VI of palbociclib may be dried by drying under reduced pressure, by air drying, or by vacuum tray drying.

A fifteenth aspect of the present invention provides a crystalline form of palbociclib, designated as Form VII, characterized by an XRPD pattern having peaks at d-spacings of about 3.6, 4.0, 4.2, 5.7, and 8.7 Å, and further characterized by additional peaks at d-spacings of about 3.7, 3.8, 4.3, 4.7, and 5.0 Å.

Table 7 provides the d-spacing values (Å), the corresponding 2θ values, and the relative intensity of the crystalline Form VII of palbociclib.

TABLE 7

| d-spacing | Position | Relative Intensity |
|---|---|---|
| (Å) | (±0.2° 2θ) | (%) |
| 19.8 | 4.5 | 13.5 |
| 11.1 | 8.0 | 13.0 |
| 9.9 | 8.9 | 3.9 |
| 8.7 | 10.2 | 100.0 |
| 6.6 | 13.4 | 26.2 |
| 5.7 | 15.6 | 36.7 |
| 5.5 | 16.1 | 26.7 |
| 5.2 | 17.2 | 18.3 |
| 5.0 | 17.8 | 33.2 |
| 4.7 | 19.1 | 34.2 |
| 4.4 | 20.4 | 25.8 |
| 4.3 | 20.8 | 35.7 |
| 4.2 | 21.3 | 48.3 |
| 4.0 | 22.3 | 53.1 |
| 3.8 | 23.6 | 33.6 |
| 3.7 | 24.1 | 31.0 |
| 3.6 | 25.0 | 40.5 |
| 3.5 | 25.7 | 28.2 |
| 3.3 | 27.2 | 14.8 |
| 3.2 | 28.3 | 7.7 |
| 3.0 | 29.5 | 18.1 |
| 2.9 | 31.4 | 6.7 |
| 2.7 | 33.2 | 7.2 |
| 2.6 | 35.2 | 3.5 |
| 2.5 | 36.1 | 5.5 |

Crystalline Form VII of palbociclib is characterized by a DSC thermogram having endothermic peaks at about 98.5° C., 251.2° C., 269.3° C., and 285.0° C.

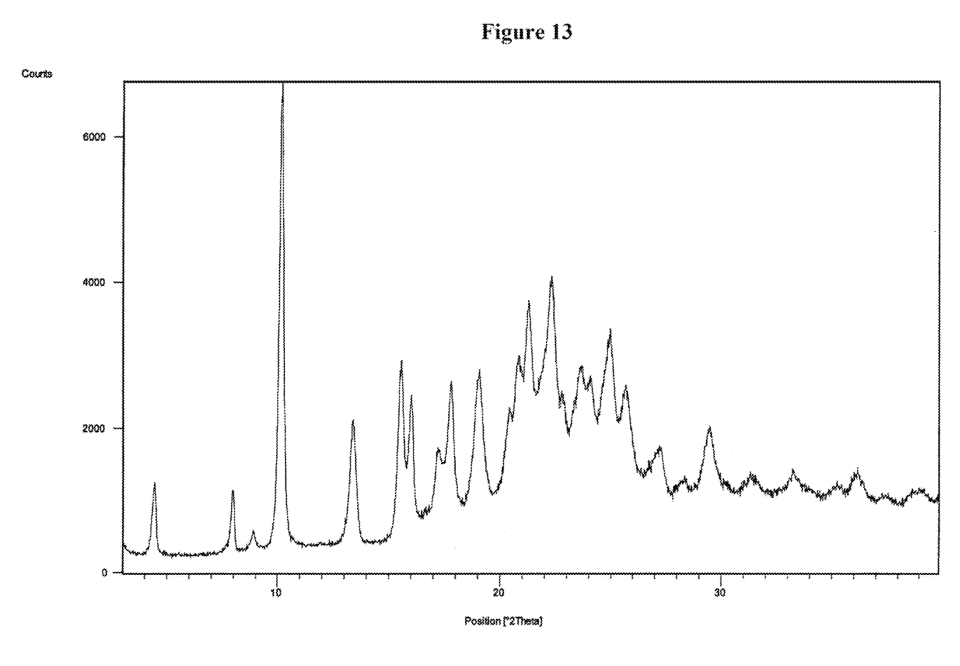

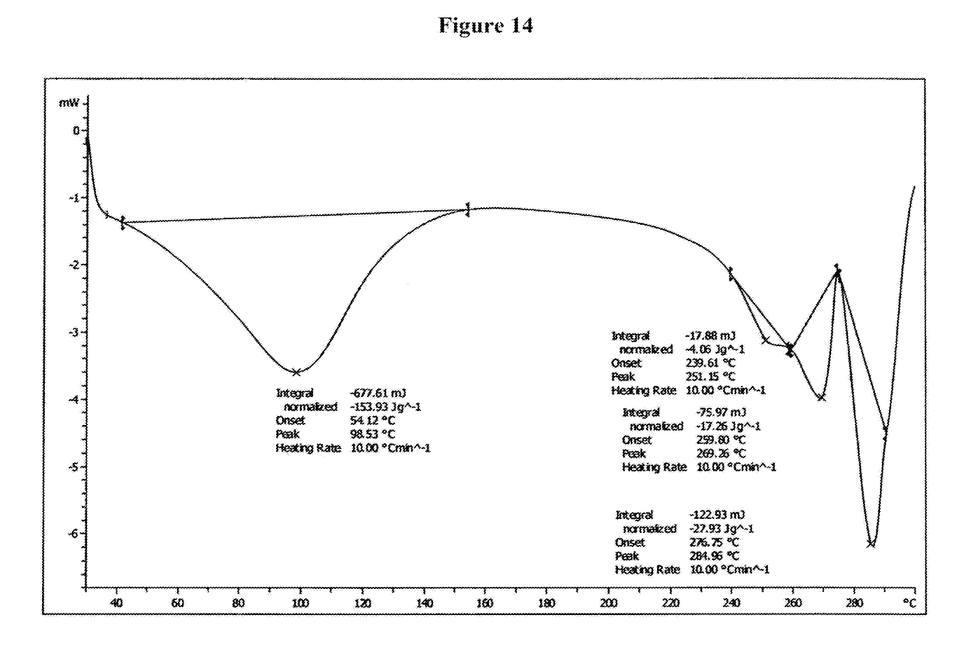

Crystalline Form VII of palbociclib is characterized by an XRPD pattern substantially as depicted in FIG. 13 or a DSC thermogram substantially as depicted in FIG. 14.

A sixteenth aspect of the present invention provides a process for the preparation of crystalline Form VII of palbociclib, comprising:
i) contacting crystalline Form III of palbociclib with a mixture of water and methanol;
ii) adding sulphuric acid to the reaction mixture of step i); and
iii) adding ammonia to the reaction mixture of step ii) to obtain crystalline Form VII of palbociclib.

The preparation of crystalline Form VII of palbociclib is carried out at ambient temperature for a period of about 30 minutes to about 5 hours, for example, for about one hour to about 4 hours.

The crystalline Form VII of palbociclib may be isolated by filtration, decantation, extraction, distillation, evaporation, chromatography, precipitation, concentration, crystallization, centrifugation, or recrystallization. The crystalline Form VII of palbociclib may be dried by drying under reduced pressure, by air drying, or by vacuum tray drying.

A seventeenth aspect of the present invention provides a crystalline form of palbociclib, designated as Form VIII, characterized by an XRPD pattern having peaks at d-spacings of about 2.7, 3.9, 4. 0, 4.7, and 4.8 Å and further characterized by additional peaks at d-spacings of about 4.2, 4.5, 5.3, 7.7, and 8.7 Å.

Table 8 provides the d-spacing values (Å), the corresponding 2θ values, and the relative intensity of the crystalline Form VIII of palbociclib.

TABLE 8

| d-spacing | Position | Relative Intensity |
|---|---|---|
| (Å) | (±0.2° 2θ) | (%) |
| 17.4 | 5.1 | 11.0 |
| 11.2 | 7.9 | 10.9 |
| 10.5 | 8.5 | 2.9 |
| 9.3 | 9.5 | 6.7 |
| 8.7 | 10.2 | 34.8 |
| 7.7 | 11.4 | 31.4 |
| 6.7 | 13.1 | 2.9 |
| 6.3 | 14.0 | 3.9 |
| 5.9 | 15.1 | 6.9 |
| 5.6 | 15.7 | 12.4 |
| 5.4 | 16.4 | 16.3 |
| 5.3 | 16.9 | 34.6 |
| 4.8 | 18.6 | 35.7 |
| 4.7 | 19.0 | 36.2 |
| 4.5 | 19.6 | 27.8 |
| 4.4 | 20.3 | 15.1 |
| 4.2 | 21.0 | 23.9 |
| 4.0 | 22.2 | 100.0 |
| 3.9 | 22.9 | 42.1 |
| 3.8 | 23.7 | 20.4 |
| 3.5 | 25.8 | 15.1 |
| 3.3 | 27.2 | 11.6 |
| 3.2 | 28.2 | 19.7 |
| 3.0 | 29.8 | 9.6 |
| 2.9 | 30.7 | 9.1 |
| 2.8 | 31.7 | 9.9 |
| 2.7 | 32.7 | 64.4 |
| 2.6 | 34.7 | 2.9 |

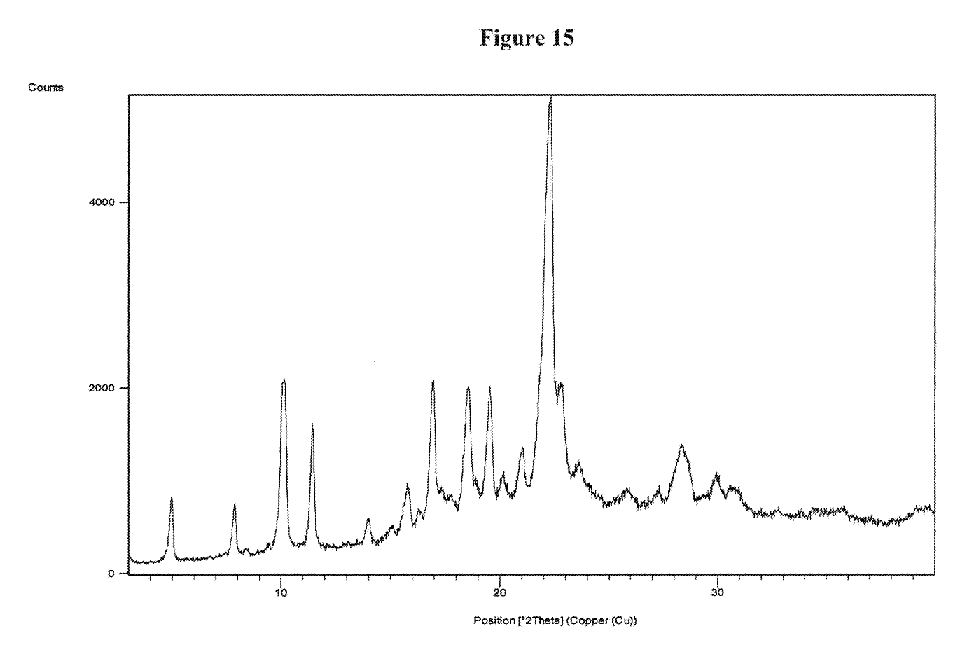

The crystalline Form VIII of palbociclib is characterized by an XRPD pattern substantially as depicted in FIG. 15.

An eighteenth aspect of the present invention provides a process for the preparation of crystalline Form VIII of palbociclib, comprising the steps of:
i) contacting crystalline Form I of palbociclib with a mixture of water and dimethyl formamide;
ii) adding an acid to the reaction mixture of step i); and
iii) adding a base to the reaction mixture of step ii) to obtain crystalline Form VIII of palbociclib.

Examples of acids include hydrochloric acid and sulphuric acid.

Examples of bases include ammonia and sodium bicarbonate.

The preparation of crystalline Form VIII of palbociclib is carried out at ambient temperature for a period of about 30 minutes to about 5 hours, for example, for about one hour to about 3 hours.

Crystalline Form VIII of palbociclib may be isolated by filtration, decantation, extraction, distillation, evaporation, chromatography, precipitation, concentration, crystallization, centrifugation, or recrystallization. Crystalline Form VIII of palbociclib may be dried by drying under reduced pressure, by air drying, or by vacuum tray drying.

A nineteenth aspect of the present invention provides a pharmaceutical composition comprising crystalline forms selected from the group consisting of Form I, Form II, Form III, Form IV, Form V, Form V-A, Form VI, Form VII, and Form VIII of palbociclib, and one or more pharmaceutically acceptable carriers, diluents, or excipients.

A twentieth aspect of the present invention provides a method for treating cyclin-dependent kinase associated diseases comprising administering to a patient in need thereof a therapeutically effective amount of a composition comprising crystalline forms selected from the group consisting of Form I, Form II, Form III, Form IV, Form V, Form V-A, Form VI, Form VII, and Form VIII of palbociclib.

While the present invention has been described in terms of its specific aspects and embodiments, certain modifications and equivalents will be apparent to those skilled in the art, and are intended to be included within the scope of the present invention.

### Methods

XRPD of the samples was determined by using a PANalytical® instrument; Model X'pert PRO; Detector: X'celerator®.

IR of the samples was recorded using a PerkinElmer® instrument, potassium bromide pellet method.

DSC of the samples was recorded using a Mettler-Toledo® 821e instrument.

TGA was recorded using a TA Instruments® Q500.

SEM analysis was carried out using a JEOL® JSM-6010LV instrument. The samples were coated by a JEOL® Platinum Sputter Coater.

Particle size distribution was measured using a Malvern® Mastersizer® 2000 instrument.

Specific surface area was determined using a Micromeritic® Gemini® VII 2390 Surface Analyzer (Software: Gemini VII, version 1.03).

The following examples are for illustrative purposes only and should not be construed as limiting the scope of the invention in any way.

### EXAMPLES

### Example 1: Preparation of Palbociclib Hydrochloride

Concentrated hydrochloric acid (13 mL) was added to a solution of tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate (15 g) (obtained per the procedure disclosed in U.S. Pat. No. 7,781,583) in methanol (150 mL) at 25° C. to 30° C. to obtain a reaction mixture. The reaction mixture was heated to 50° C. to 55° C. for 2 hours, then cooled to ambient temperature. Sodium hydroxide solution (20%, 20 mL) was added to the reaction mixture to adjust the pH to 7.5 to 8.0 to obtain a solid. The solid was stirred at 25° C. to 30° C. for one hour. The solid was filtered, then washed with water (30 mL), and then washed with methanol (30 mL) to obtain the title compound as a light grey solid.

Yield: 14 g

### Example 2: Preparation of a Crystalline Form I of Palbociclib

Palbociclib hydrochloride (14 g, as obtained in Example 1) was suspended in a mixture of water and methanol (1:1, 160 mL) at 25° C. to 30° C. to obtain a reaction mixture. Concentrated hydrochloric acid (25 mL) was added to the reaction mixture to obtain a clear solution. Activated carbon (1.4 g) was added to the reaction mixture and the mixture was stirred for 10 minutes to 15 minutes. The reaction mixture was filtered through a Hyflo® bed and then washed with a mixture of water and methanol (1:1, 28 mL) to obtain a filtrate. Sodium hydroxide (20%, 35 mL) was added to the filtrate to adjust the pH to 7.5 to 8.0 to obtain a solid. The solid was stirred for 45 minutes to 60 minutes at 25° C. to 30° C. The solid was filtered, then washed with a mixture of water and methanol (1:1, 40 mL), and then dried under vacuum at 35° C. to 40° C. for 8 hours to 10 hours to obtain the title compound.

Yield: 10 g

### Example 3: Preparation of a Crystalline Form II of Palbociclib

Method A:

Palbociclib (1 g, Form I as obtained in Example 2) was suspended in a mixture of water and acetone (1:1, 10 mL) at 25° C. to 30° C. to obtain a reaction mixture. Concentrated hydrochloric acid (6 mL) was added to the reaction mixture and the mixture was stirred for 5 minutes to 10 minutes. Activated carbon (100 mg) was added to the reaction mixture and the mixture was stirred for 10 minutes. The reaction mixture was filtered, and then washed with a mixture of water and acetone (1:1, 4 mL) to obtain a filtrate. Sodium hydroxide (20%, 12 mL) was added to the filtrate to adjust the pH to 7.5 to 8.0 to obtain a solid. The solid was stirred for 20 minutes to 30 minutes at 25° C. to 30° C. The solid was filtered, then washed with a mixture of water and acetone (1:1, 10 mL), and then dried under vacuum at 35° C. to 40° C. for 10 hours to 12 hours to obtain the title compound.

Yield: 0.65 g

Method B:

Palbociclib (1 g, Form I as obtained in Example 2) was suspended in a mixture of water and 2-propanol (1:1, 10 mL) at 25° C. to 30° C. to obtain a reaction mixture. Concentrated hydrochloric acid (7 mL) was added to the reaction mixture and the mixture was stirred for 5 minutes to 10 minutes at 25° C. to 30° C. Activated carbon (100 mg) was added to the reaction mixture and the mixture was stirred for 10 minutes. The reaction mixture was filtered through a Hyflo® bed, and then washed with a mixture of water and 2-propanol (1:1, 4 mL) to obtain a filtrate. Sodium hydroxide (20%, 15 mL) was added to the filtrate to adjust the pH to 9.0 to 9.5 to obtain a solid. The solid was stirred for 20 minutes to 30 minutes at 25° C. to 30° C. The solid was filtered, then washed with a mixture of water and 2-propanol (1:1, 10 mL), and then dried under vacuum at 35° C. to 40° C. for 10 hours to 12 hours to obtain the title compound.

Yield: 0.5 g

### Example 4: Preparation of a Crystalline Form III of Palbociclib

Method A:

Palbociclib (0.3 g, Form I as obtained in Example 2) was suspended in a mixture of water and 2-propanol (1:1, 20 mL) at 25° C. to 30° C. to obtain a reaction mixture. Concentrated hydrochloric acid (100 μL) was added to the reaction mixture and the mixture was stirred for 5 minutes. Aqueous ammonia (25%, 500 μL) was added to the reaction mixture and the mixture was stirred for 25 minutes to obtain a solid. The solid was filtered, then washed with water (5 mL), and then dried under vacuum at 25° C. to 30° C. for 18 hours to obtain the title compound.

Yield: 0.19 g

Method B:

Palbociclib (0.3 g, Form I as obtained in Example 2) was suspended in a mixture of water and acetonitrile (1:1, 10 mL) at 25° C. to 30° C. to obtain a reaction mixture. Concentrated hydrochloric acid (300 μL) was added to the reaction mixture and the mixture was stirred for 2 minutes to 3 minutes. Aqueous ammonia (25%, 500 μL) was added to the reaction mixture and the mixture was stirred for one hour to obtain a solid. The solid was filtered, then washed with water (5 mL), and then dried under vacuum at 25° C. to 30° C. for 16 hours to obtain the title compound.

Yield: 0.187 g

Method C:

Palbociclib (0.3 g, Form I as obtained in Example 2) was suspended in a mixture of water and acetone (1:1, 14 mL) at 25° C. to 30° C. to obtain a reaction mixture. Concentrated hydrochloric acid (100 μL) was added to the reaction mixture and the mixture was stirred for 2 minutes to 3 minutes. Liquid ammonia (25%, 500 μL) was added to the reaction mixture and the mixture was stirred for one hour to obtain a solid. The solid was filtered, then washed with water (5 mL), and then dried under vacuum at 25° C. to 30° C. for 16 hours to obtain the title compound.

Yield: 0.197 g

### Example 5: Preparation of a Crystalline Form IV of Palbociclib

Palbociclib (0.3 g, Form I as obtained in Example 2) was suspended in a mixture of water and 1-propanol (1:1, 20 mL) at 25° C. to 30° C. to obtain a reaction mixture. Concentrated hydrochloric acid (100 μL) was added to the reaction mixture and the mixture was stirred for 5 minutes. Aqueous ammonia (25%, 500 μL) was added to the reaction mixture and the mixture was stirred for 10 minutes to obtain a solid. The solid obtained was filtered, then washed with water (5 mL), and then dried under vacuum at 25° C. to 30° C. for 17 hours to obtain the title compound.

Yield: 0.185 g

### Example 6: Preparation of a Crystalline Form V of Palbociclib

Method A:

Palbociclib (1 g, Form III as obtained in Example 4) was suspended in a mixture of water and methanol (1:1, 36 mL) at 25° C. to 30° C. to obtain a reaction mixture. Dilute sulphuric acid (2:1 ratio of H₂O:H₂SO₄) was added drop-wise to the reaction mixture until the pH was about 2 to 3. The reaction mixture was stirred for 20 minutes at 25° C. to 30° C. Saturated sodium bicarbonate solution in water was added to the reaction mixture until the pH was 7 to 8. The reaction mixture was stirred for 3 hours to 4 hours at 25° C. to 30° C. to obtain a solid. The solid was filtered, then washed with deionized (DI) water (22 mL), and then dried under vacuum at 50° C. for 12 hours to obtain the title compound.

Yield: 0.9 g

Method B:

Palbociclib (1 g, Form III as obtained in Example 4) was suspended in a mixture of water and methanol (1:1, 36 mL) at 25° C. to 30° C. to obtain a reaction mixture. Dilute sulphuric acid (2:1 ratio of H₂O:H₂SO₄) was added drop-wise to the reaction mixture until the pH was about 2 to 3. The reaction mixture was stirred for 10 minutes at 25° C. to 30° C. Saturated sodium bicarbonate solution in water was added to the reaction mixture until the pH was 7 to 8. The reaction mixture was stirred for 30 minutes at 25° C. to 30° C. to obtain a solid. The solid obtained was filtered, then washed with DI water (22 mL), and then dried under vacuum at 50° C. for 9 hours to obtain the title compound.

Yield: 0.85 g

Method C:

Palbociclib (1 g, Form III as obtained in Example 4) was suspended in a mixture of water and methanol (1:1, 36 mL) at 25° C. to 30° C. to obtain a reaction mixture. Dilute sulphuric acid (2:1 ratio of H₂O:H₂SO₄) was added drop-wise to the reaction mixture until the pH was about 2 to 3. The reaction mixture was stirred for 10 minutes at 25° C. to 30° C. Saturated sodium bicarbonate solution in water was added to the reaction mixture until the pH was 7 to 8. The reaction mixture was stirred for 4 hours at 25° C. to 30° C. to obtain a solid. The solid obtained was filtered, then washed with DI water (22 mL), and then dried under vacuum at 50° C. for 9 hours to obtain the title compound.

Yield: 0.95 g

Method D:

Palbociclib (0.1 g, Form III as obtained in Example 4) was suspended in a mixture of water and methanol (1:1, 4 mL) at 25° C. to 30° C. to obtain a reaction mixture. Dilute phosphoric acid (1:1 ratio of H₂O:H₃PO₄) was added drop-wise to the reaction mixture until the pH was about 2 to 3. The reaction mixture was stirred for one hour at 25° C. to 30° C. Saturated sodium bicarbonate solution in water was added to the reaction mixture until the pH was 7 to 8. The reaction mixture was stirred for 3.5 hours at 25° C. to 30° C. to obtain a solid. The solid obtained was filtered, then dried under vacuum at 50° C. for 6 hours to obtain the title compound.

Yield: 0.09 g

Method E:

Palbociclib (0.1 g, Form III as obtained in Example 4) was suspended in a mixture of water and methanol (1:1, 4 mL) at 25° C. to 30° C. to obtain a reaction mixture. Dilute phosphoric acid (1:1 ratio of H₂O:H₃PO₄) was added drop-wise to the reaction mixture until the pH was about 2 to 3. The reaction mixture was stirred for one hour at 25° C. to 30° C. Saturated potassium carbonate solution in water was added to the reaction mixture until the pH was 7 to 8. The reaction mixture was stirred for 3.5 hours at 25° C. to 30° C. to obtain a solid. The solid obtained was filtered, then dried under vacuum at 50° C. for 6 hours to obtain the title compound.

Yield: 0.07 g

### Example 7: Preparation of a Crystalline Form V-A of Palbociclib

Palbociclib hydrochloride (100 g, as obtained in Example 1) was dissolved in a mixture of water and methanol (1:1, 4000 mL) at 25° C. to 30° C. to obtain a reaction mixture. Eno anti chromos carbon (20 g) and sodium metabisulphite (2 g) were added to the reaction mixture and the mixture was stirred for 30 minutes. The reaction mixture was filtered through a Hyflo® bed and then washed with water (500 mL) to obtain a filtrate. The filtrate was passed through a 0.45 micron filter. The filtrate obtained was heated at 40° C. to 50° C. The filtrate was treated with aqueous sodium bicarbonate (5%) to adjust the pH to 7.0 to 7.2 over 90 minutes at 40° C. to 50° C. The reaction mixture was stirred at 40° C. to 50° C. for 2 hours to 3 hours. The reaction mixture was filtered, and then washed with water (2×200 mL), methanol (lx 200 mL) and acetone (lx 200 mL) to obtain a solid. The solid obtained was dried at 50° C. to 60° C. for 12 hours to obtain the title compound.

Yield: 75 g

### Example 8: Preparation of a Crystalline Form VI of Palbociclib

Method A:

Palbociclib (0.15 g, Form III as obtained in Example 4) was suspended in 2-propanol (0.5 mL) to obtain a slurry. The slurry was stirred at 45° C. for 2 hours on a Rotavapor®. The solvent was dried under vacuum at room temperature for 5 hours to obtain a solid. The solid was further dried under vacuum at 50° C. for 12 hours to obtain the title compound.

Yield: 0.1 g

Method B:

Palbociclib (0.15 g, Form III as obtained in Example 4) was suspended in acetone (0.5 mL) to obtain a slurry. The slurry was stirred at 45° C. for 2 hours on a Rotavapor®. The solvent was dried under vacuum at room temperature for 5 hours to obtain a solid. The solid was further dried under vacuum at 50° C. for 12 hours to obtain the title compound.

Yield: 0.11 g

Method C:

Palbociclib (0.15 g, Form III as obtained in Example 4) was suspended in tetrahydrofuran (0.5 mL) to obtain a slurry. The slurry was stirred at 45° C. for 2 hours on a Rotavapor®. The solvent was dried under vacuum at room temperature for 5 hours to obtain a solid. The solid was further dried under vacuum at 50° C. for 12 hours to obtain the title compound.

Yield: 0.1 g

Method D:

Palbociclib (0.15 g, Form III as obtained in Example 4) was suspended in 2-propylacetate (0.5 mL) to obtain a slurry. The slurry was stirred at 45° C. for 2 hours on a Rotavapor®. The solvent was dried under vacuum at room temperature for 5 hours to obtain a solid. The solid was dried further under vacuum at 50° C. for 12 hours to obtain the title compound.

Yield: 0.1 g

### Example 9: Preparation of a Crystalline Form VII of Palbociclib

Palbociclib (1.5 g, Form III as obtained in Example 4) was suspended in a mixture of water and methanol (1:1, 50 mL) at 25° C. to 30° C. to obtain a reaction mixture. Dilute sulphuric acid (1200 μL) was added drop-wise to the reaction mixture until the pH was 2 to 3. The reaction mixture was stirred for one hour at 25° C. to 30° C. Aqueous ammonia was added to the reaction mixture until the pH was 10 to 11. The reaction mixture was stirred for 3.5 hours at 25° C. to 30° C. to obtain a solid. The solid obtained was filtered, then dried under vacuum at 50° C. for 6 hours to obtain the title compound.

Yield: 1.5 g

### Example 10: Preparation of a Crystalline Form VIII of Palbociclib

Palbociclib (0.3 g, Form I as obtained in Example 2) was suspended in a mixture of water and dimethyl formamide (1:1, 10 mL) at 25° C. to 30° C. to obtain a reaction mixture. Concentrated hydrochloric acid (500 μL) was added to the reaction mixture and the mixture was stirred for 2 minutes to 3 minutes at 25° C. to 30° C. Aqueous ammonia (25%, 800 μL) was added to the reaction mixture and then the reaction mixture was stirred at 25° C. to 30° C. for 30 minutes to obtain a solid. The solid obtained was filtered, then washed with water (5 mL), and then dried under vacuum at 25° C. to 30° C. for 7 hours to obtain the title compound.

Yield: 0.193 g

1. Crystalline Form VA of palbociclib characterized by
an X-ray powder diffraction (XRPD) pattern having peaks at d-spacings of about 11.3, 6.8, 5.3, 4.2, and 3.7 Å; or
a differential scanning calorimetry (DSC) thermogram having endothermic peaks at about 148.2° C., 269.2° C., 272.1° C., and 284.2° C. and an exothermic peak at about 222.9° C.; or
an Infra-red (IR) absorption spectrum having characteristic peaks at about 3422, 3235, 3168, 2948, 2865, 2804, 2468, 1692, 1654, 1585, 1555, 1488, 1454, 1399, 1377, 1349, 1315, 1293, 1279, 1263, 1233, 1160, 1143, 1125, 1083, 1040, 1016, 990, 931, 906, 826, 801, 749, 724, 691, 635, 624, 567, 543, 445, and 431 cm⁻¹; or
specific surface area greater than 2 m²/g; or
particle size distribution of at least one of: aa) a D10 value of about 1 μm to about 5 μm; bb) a D50 value of about 5 μm to about 20 μm; or cc) a D90 value of about 15 μm to about 50 μm; or
a volume mean diameter (D[4,3]) of about 5 μm to about 50 μm; or
an XRPD pattern substantially as depicted in FIG. 16; or
a DSC thermogram substantially as depicted in FIG. 17; or
an IR absorption substantially as depicted in FIG. 18; or
a thermogravimetric analysis (TGA) thermogram substantially as depicted in FIG. 19; or
a scanning electron microscopy (SEM) image substantially as depicted in FIG. 20.

2.-13. (canceled)

14. A process for the preparation of the crystalline Form VA of claim 1, comprising
i) contacting palbociclib hydrochloride with water and mixture of methanol or acetone; and
ii) adding an inorganic base to the reaction mixture of step i) to obtain the crystalline Form V of palbociclib.

15. (canceled)

16. Crystalline Form I of palbociclib, characterized by
an X-ray powder diffraction (XRPD) pattern having peaks at d-spacings of about 2.8, 3.3, 3.6, 3.9, and 7.2 Å; or
a differential scanning calorimetry (DSC) thermogram having endothermic peaks at about 81.3° C., 155.2° C., 238.0° C., and 265.3° C. and an exothermic peak at 185.7° C.; or
an infra-red (IR) absorption spectrum having characteristic peaks at about 3420.2, 3233.7, 2953.9, 2481.6, 1696.9, 1649.5, 1580.9, 1552.2, 1454.5, 1400.0, 1371.7, 1289.4, 1250.4, 1151.1, 1122.0, 1084.4, 1017.6, 923.8, 821.5, 802.0, 745.8, 722.7, 689.0, 631.5, 562.0, and 465.8 cm⁻¹; or
an XRPD pattern substantially as depicted in FIG. 1: or
a DSC thermogram substantially as depicted in FIG. 2; or
an IR absorption spectrum substantially as depicted in FIG. 3.

17.-22. (canceled)

23. A process for the preparation of the crystalline Form I of claim 16, comprising:
i) contacting palbociclib hydrochloride with a mixture of methanol and water;
ii) adding an acid to the reaction mixture of step i); and
iii) adding a base to the reaction mixture of step ii) to obtain the crystalline Form I of palbociclib.

24.-25. (canceled)

26. Crystalline Form II of palbociclib, characterized by
an XRPD pattern having peaks at d-spacings of about 2.8, 4.0, 4.5, 5.2, and 8.6 Å; or
a DSC thermogram having an endothermic peak at about 78.0° C. and an exothermic peak at about 275.1° C.; or
an IR absorption spectrum having characteristic peaks at about 3417, 3298, 3232, 3173, 3084, 2947, 2869, 2843, 2469, 1770, 1664, 1602, 1581, 1548, 1528, 1488, 1449, 1396, 1366, 1332, 1310, 1284, 1248, 1235, 1188, 1148, 1122, 1078, 1039, 1022, 978, 937, 924, 899, 873, 860, 827, 805, 795, 776, 758, 747, 737, 720, 689, 646, 627, 617, 572, 533, 457, 431, and 407 cm⁻¹; or
an XRPD pattern substantially as depicted in FIG. 4; or
a DSC thermogram substantially as depicted in FIG. 5; or
an IR spectrum as depicted in FIG. 6.

27.-32. (canceled)

33. A process for the preparation of the crystalline Form II of claim 26 comprising:
i) contacting crystalline Form I of palbociclib with a mixture of solvents selected from the group consisting of water and acetone, and water and 2-propanol;
ii) adding hydrochloric acid to the reaction mixture of step i); and
iii) adding sodium hydroxide to the reaction mixture of step ii) to obtain the crystalline Form II of palbociclib.

34. Crystalline Form III of palbociclib, characterized by
an XRPD pattern having peaks at d-spacings of 4.0, 5.2, 8.6, and 8.8 Å; or
an XRPD pattern having additional peaks at d-spacings of 3.2, 3.9, 4.5, 4.8, and 7.7 Å; or
an XRPD pattern as depicted in FIG. 7.

35.-36. (canceled)

37. A process for the preparation of crystalline Form III of claim 34, comprising:
i) contacting crystalline Form I of palbociclib with a mixture of solvents selected from the group consisting of water and 2-propanol, water and acetonitrile, and water and acetone;
ii) adding hydrochloric acid to the reaction mixture of step i); and
iii) adding ammonia to the reaction mixture of step ii) to obtain the crystalline Form III of palbociclib.

38. Crystalline Form IV of palbociclib, characterized by an XRPD pattern having peaks at d-spacings of about 4.4, 5.8, 8.7, 11.1, and 17.3 Å; or an XRPD pattern substantially as depicted in FIG. 8.

39.-40. (canceled)

41. A process for the preparation of crystalline Form IV of claim 38, comprising:
i) contacting crystalline Form I of palbociclib with a mixture of water and 1-propanol;
ii) adding hydrochloric acid to the reaction mixture of step i); and
iii) adding ammonia to the reaction mixture of step ii) to obtain the crystalline Form IV of palbociclib.

42. Crystalline Form V of palbociclib, characterized by
an XRPD pattern having peaks at d-spacings of about 4.2, 5.4, 10.0, 11.3, and 15.6 Å; or
a DSC thermogram having endothermic peaks at about 62.6° C., 126.0° C., and 262.8° C.; or
an IR absorption spectrum having characteristic peaks at about 3418.3, 3231.6, 3166.9, 2947.6, 2865.3, 1691.1, 1655.1, 1585.3, 1552.9, 1489.6, 1453.6, 1398.7, 1376.3, 1349.8, 1315.8, 1283.3, 1233.7, 1159.8, 1122.3, 1082.4, 1039.7, 1015.6, 990.3, 929.9, 906.5, 825.9, 800.9, 748.4, 723.8, 691.0, 623.7, 564.9, 542.6, 447.8, and 430.2 cm⁻¹ or
an XRPD pattern substantially as depicted in FIG. 9; or
by a DSC thermogram substantially as depicted in FIG. 10; or
an IR absorption spectrum substantially as depicted in FIG. 11.

43.-48. (canceled)

49. A process for the preparation of crystalline Form V of claim 42 comprising:
i) contacting crystalline Form III of palbociclib with a mixture of water and methanol;
ii) adding an inorganic acid selected from the group consisting of sulphuric acid, and phosphoric acid to the reaction mixture of step i); and
iii) adding an inorganic base selected from the group consisting of sodium bicarbonate and potassium carbonate to the reaction mixture of step ii) to obtain the crystalline Form V of palbociclib.

50. Crystalline Form VI of palbociclib, characterized by
an XRPD pattern having peaks at d-spacings of about 3.5, 3.6, 3.7, 4.0, and 4.3 Å; or
an XRPD pattern substantially as depicted in FIG. 12.

51.-52. (canceled)

53. A process for the preparation of crystalline Form VI of claim 50 comprising contacting crystalline Form III of palbociclib with a solvent selected from the group consisting of 2-propanol, acetone, tetrahydrofuran, and 2-propyl acetate to obtain the crystalline Form VI of palbociclib.

54. Crystalline Form VII of palbociclib, characterized by
an XRPD pattern having peaks at d-spacings of about 3.6, 4.0, 4.2, 5.7, and 8.7 Å; or
a DSC thermogram having endothermic peaks at about 98.5° C., 251.2° C., 269.3° C., and 285.0° C.; or
an XRPD pattern substantially as depicted in FIG. 13; or
a DSC thermogram substantially as depicted in FIG. 14.

55.-58. (canceled)

59. A process for the preparation of crystalline Form VII of claim 54 comprising:
i) contacting crystalline Form III of palbociclib with a mixture of water and methanol;
ii) adding sulphuric acid to the reaction mixture of step i); and
iii) adding ammonia to the reaction mixture of step ii) to obtain the crystalline Form VII of palbociclib.

60. Crystalline Form VIII of palbociclib, characterized by an XRPD pattern having peaks at d-spacings of about 2.7, 3.9, 4.0, 4.7, and 4.8 Å; or
an XRPD pattern substantially as depicted in FIG. 15.

61.-62. (canceled)

63. A process for the preparation of crystalline Form VIII of claim 60, comprising:
i) contacting crystalline Form I of palbociclib with a mixture of water and dimethyl formamide;
ii) adding an acid to the reaction mixture of step i); and
iii) adding a base to the reaction mixture of step ii) to obtain the crystalline Form VIII of palbociclib.

64.-67. (canceled)
